(19) United States
(12) Patent Application Publication
(10) Pub. No.: US 2020/0058391 A1
(43) Pub. Date: Feb. 20, 2020

(54) DYNAMIC SYSTEM FOR DELIVERING FINDING-BASED RELEVANT CLINICAL CONTEXT IN IMAGE INTERPRETATION ENVIRONMENT

(71) Applicant: KONINKLIJKE PHILIPS N.V., EINDHOVEN (NL)

(72) Inventors: Merlijn SEVENSTER, HAARLEM (NL); Amir Mohammad TAHMASEBI MARAGHOOSH, ARLINGTON, MA (US); Kirk SPENCER, CHICAGO, IL (US)

(21) Appl. No.: 16/610,251

(22) PCT Filed: Apr. 25, 2018
(86) PCT No.: PCT/EP2018/060513

§ 371 Date: Nov. 1, 2019

Related U.S. Application Data
(60) Provisional application No. 62/501,853, filed on May 5, 2017.

(51) Int. Cl. G16H 30/40; G16H 10/60; G16H 15/00; G06K 9/32

(52) U.S. Cl. CPC G16H 30/40; G06K 9/325; G16H 15/00; G16H 10/60

(57) ABSTRACT

An image interpretation workstation includes a display (12), user input devices (14, 16, 18), an electronic processor (20, 22), and a non-transitory storage medium storing instructions readable and executable by the electronic processor. An image interpretation environment (31) is implemented by instructions (30) which display medical images on the at least one display, manipulate the displayed medical images, generate finding objects, and construct of an image examination findings report (40). Finding object detection instructions (32) monitor the image interpretation environment to detect generation of a finding object or user selection of a finding object. Patient record retrieval instructions (34) identify and retrieve patient information relevant to a finding object (40) detected by the finding object detection instructions from at least one electronic patient record (24, 25, 26). Patient record display instructions (36) display patient information retrieved by the patient record retrieval instructions on the at least one display.

### FIELD

The following relates generally to the image interpretation workstation arts, radiology arts, echocardiography arts, and related arts.

### BACKGROUND

An image interpretation workstation provides a medical professional such as a radiologist or cardiologists with the tools to view images, manipulate images by operations such as pan, zoom, three-dimensional (3D) rendering or projection, and so forth, and also provides the user interface for selecting and annotating portions of the images and for generating an image examination findings report. As an example, in a radiology examination workflow, a radiology examination is ordered and the requested images are acquired using a suitable imaging device, e.g. a magnetic resonance imaging (MRI) device for MR imaging, a positron emission tomography (PET) imaging device for PET imaging, a gamma camera for single photon emission computed tomography (SPECT) imaging, a transmission computed tomography (CT) imaging device for CT imaging, or so forth. The medical images are typically stored in a Picture Archiving and Communication System (PACS), or in a specialized system such as a cardiovascular information system (CVIS). After the actual imaging examination, a radiologist operating a radiology interpretation workstation retrieves the images from the PACS, reviews them on the display of the workstation, and types, dictates, or otherwise generates a radiology findings report.

As another illustrative workflow example, an echocardiogram is ordered, and an ultrasound technician or other medical professional acquires the requested echocardiogram images. A cardiologist or other professional operating an image interpretation workstation retrieves the echocardiogram images, reviews them on the display of the workstation, and types, dictates, or otherwise generates an echocardiogram findings report.

In such imaging examinations, the radiologist, cardiologist, or other medical professional performing the image interpretation can benefit from reviewing the patient's medical record (i.e. patient record), which may contain information about the patient that is informative in drawing appropriate clinical findings from the images. The patient's medical record is preferably stored electronically in an electronic database such as an electronic medical record (EMR), an electronic health record (EHR), or in a domain-specific electronic database such as the aforementioned CVIS for cardiovascular treatment facilities. To this end, it is known to provide access to the patient record stored at the EMR, EHR, CVIS, or other database via the image interpretation workstation. For example, the image interpretation environment may execute as one program running on the workstation, and the EMR interface may execute as a second program running concurrently on the workstation.

The following discloses certain improvements.

### SUMMARY

In one disclosed aspect, an image interpretation workstation comprises at least one display, at least one user input device, an electronic processor operatively connected with the at least one display and the at least one user input device, and a non-transitory storage medium storing instructions readable and executable by the electronic processor. Image interpretation environment instructions are readable and executable by the electronic processor to perform operations in accord with user inputs received via the at least one user input device including display of medical images on the at least one display, manipulation of displayed medical images, generation of finding objects, and construction of an image examination findings report. Finding object detection instructions are readable and executable by the electronic processor to detect generation of a finding object or user selection of a finding object via the at least one user input device. Patient record retrieval instructions are readable and executable by the electronic processor to identify and retrieve patient information relevant to a finding object detected by the finding object detection instructions from at least one electronic patient record. Patient record display instructions are readable and executable by the electronic processor to display patient information retrieved by the patient record retrieval instructions on the at least one display.

In another disclosed aspect, a non-transitory storage medium stores instructions readable and executable by an electronic processor operatively connected with at least one display and at least one user input device to perform an image interpretation method. The method comprises: providing an image interpretation environment to perform operations in accord user inputs received via the at least one user input device including display of medical images on the at least one display, manipulation of displayed medical images, generation of finding objects, and construction of an image examination findings report; monitoring the image interpretation environment to detect generation or user selection of a finding object; identifying and retrieving patient information relevant to the generated or user-selected finding object from at least one electronic patient record; and displaying the retrieved patient information on the at least one display and in the image interpretation environment.

In another disclosed aspect, an image interpretation method is performed by an electronic processor operatively connected with at least one display and at least one user input device. The image interpretation method comprises: providing an image interpretation environment to perform operations in accord user inputs received via the at least one user input device including display of medical images on the at least one display, manipulation of displayed medical images, generation of finding objects, and construction of an image examination findings report; monitoring the image interpretation environment to detect generation or user selection of a finding object; identifying and retrieving patient information relevant to the generated or user-selected finding object from at least one electronic patient record; and displaying the retrieved patient information on the at least one display and in the image interpretation environment.

One advantage resides in automatically providing patient record content relevant to an imaging finding in response to creation or selection of that finding.

Another advantage resides in providing an image interpretation workstation with an improved user interface.

Another advantage resides in providing an image interpretation workstation with more efficient retrieval of salient patient information.

Another advantage resides in providing contextual information related to a medical imaging finding.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates an image interpretation workstation with automated retrieval of patient record information relevant to image findings.

FIGS. 2 and 3 present illustrative display examples suitably generated by the image interpretation workstation of FIG. 1.

FIG. 4 diagrammatically illustrates a process workflow for automated retrieval of patient record information relevant to image findings, which is suitably performed by the image interpretation workstation of FIG. 1.

### DETAILED DESCRIPTION

It is recognized herein that existing approaches for integrating patient record information into the image interpretation workflow have certain deficiencies. For example, providing a separate, concurrently running patient record interface program does not provide timely access to relevant patient information. To use such a patient record interface, the radiologist, physician, ultrasound specialist, or other image interpreter must recognize that the patient record may contain relevant information at a given point in the image analysis, and must know a priori the specific patient information items that are most likely to be relevant, and must know where those items are located in the electronic patient record. As to the last point, the electronic patient record may be spread across a number of different databases, e.g. the Electronic Medical/Health Record (EMR or EHR) may store general patient information, the Cardiovascular Information System (CVIS) may store information specifically relating to cardiovascular care, the Picture Archiving and Communication Service (PACS) may store radiology images and related content such as radiology reports, and so forth. The particular database(s) organization is also likely to be specific to a particular hospital, which can be confusing for an image interpreter who practices at several different hospitals.

It is further recognized herein that the generation of a finding during the image interpretation (or, in some cases, the selection of a previously generated finding) can be leveraged to provide both a practical trigger for initiating the identification and retrieval of relevant patient information from the electronic patient record, and also the informational basis for such identification and retrieval. More particularly, some image interpretation workstations provide for automated or semi-automated generation of standardized and/or structured finding objects. In some radiology workstation environments, finding objects are generated in a standardized Annotation Image Mark-up (AIM) format. Similarly, some ultrasound image interpretation environments generate finding objects in the form of standardized finding codes (FCs), i.e. standard words or phrases expressing specific image findings. The generation or user selection of such a finding object is leveraged in embodiments herein to trigger a patient record retrieval operation, and the standardized and/or structured finding object provides the informational basis for this retrieval operation. The patient record retrieval process is preferably automatically triggered by generation or user selection of a finding object, and the standardized and/or structured finding object provides a finite space of data inputs so as to enable use of a relevant patient information look-up table that maps finding objects to patient information items, thereby enabling an automated retrieval process. Preferably, the retrieved patient information is automatically presented to the image interpreter in the (same) image interpretation environment being used by the image interpreter to perform the image interpretation process. In this way, the relevant patient information in the electronic patient record is automatically retrieved and presented to the image interpreter automatically, without any additional user interactions within the image interpretation environment, thus improving the user interface and operational efficiency of the image interpretation workstation.

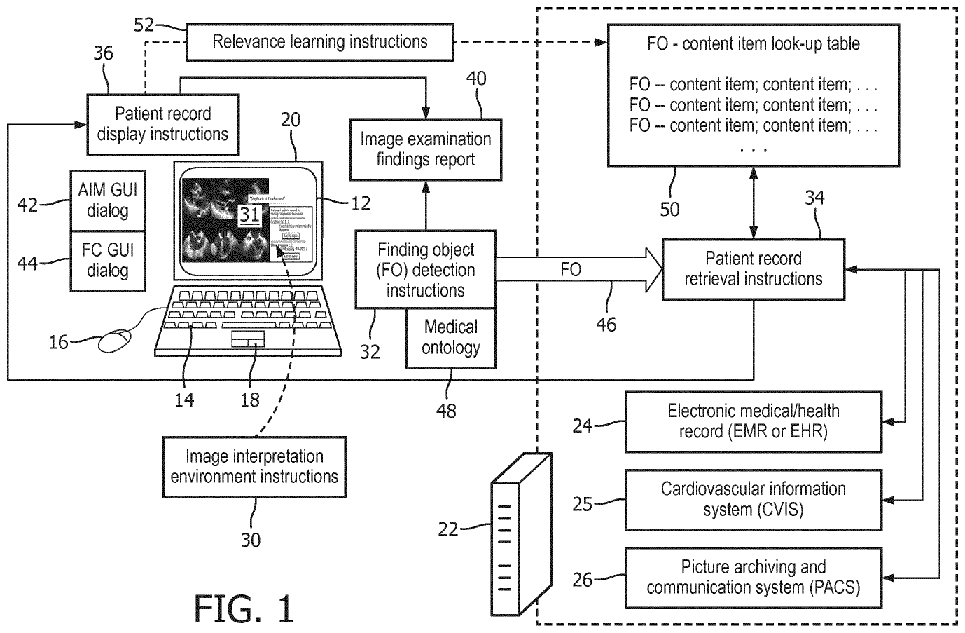

With reference to FIG. 1, an illustrative image interpretation workstation includes at least one display 12 and at least one user input device, e.g. an illustrative keyboard 14; an illustrative mouse 16, trackpad 18, trackball, touch-sensitive overlay of the display 12, or other pointing device; a dictation microphone (not shown), or so forth. The illustrative image interpretation workstation further includes electronic processors 20, 22—in the illustrative example, the electronic processor 20 is embodied for example as a local desktop or notebook computer (e.g., a local user interfacing computer) that is operated by the radiologist, ultrasound specialist, or image interpreter and include at least one microprocessor or microcontroller, and the electronic processor 22 is for example embodied as a remote server computer that is connected with the electronic processor 20 via a local area network (LAN), wireless local area network (WLAN), the Internet, various combinations thereof, and/or some other electronic data network. The electronic processor 22 optionally may itself include a plurality of interconnected computers, e.g. a computer cluster, a cloud computing resource, or so forth. The electronic processor 20, 22 includes or is in operative electronic communication with an electronic patient record 24, 25, 26, which in the illustrative embodiment is distributed across several different databases: an Electronic Medical (or Health) Record (EMR or EHR) 24 which stores general patient information; a Cardiovascular Information System (CVIS) 25 which stores information specifically relating to cardiovascular care; and a Picture Archiving and Communication Service (PACS) 26 which stores radiology images. The electronic patient record 24, 25, 26 is just one exemplary embodiment of a patient record combination and may exclude one or more of the electronic patient records 24, 25, 26, or may include additional types of electronic patient records that would otherwise be contemplated within the general nature or spirit of this disclosure (e.g., in other healthcare domains) with various permutations or combinations of electronic patient records being possible, and with the electronic patient record constituting any number of databases or even a single database. The database(s) making up the electronic patient record may have different names than those of illustrative FIG. 1, and may be specific to particular informational domains beside the illustrative general, cardiovascular, and radiology domains.

The image interpretation workstation further includes a non-transitory storage medium storing various instructions readable and executable by the electronic processor 20, 22 to perform various tasks. The non-transitory storage medium may, for example, comprise one or more of a hard disk drive or other magnetic storage medium, an optical disk or other optical storage medium, a solid state drive (SSD), FLASH memory, or other electronic storage medium, various combinations thereof, or so forth. In the illustrative embodiment, the non-transitory storage medium stores image interpretation environment instructions 30 which are readable and executable by the electronic processor 20, 22 to perform operations in accord with user inputs received via the at least one user input device 14, 16, 18 so as to implement an image interpretation environment 31. These operations include display of medical images on the at least one display 12, manipulation of displayed medical images, generation of finding objects, and construction of an image examination findings report. The image interpretation environment instructions 30 may implement substantially any suitable image interpretation environment 31, for example a radiology reading environment, an ultrasound imaging interpretation environment, a combination thereof, or so forth. A radiology reading environment is typically operatively connected with the PACS 26 to retrieve images of radiology examinations and to enable entry of an image examination findings report, sometimes referred to as a radiology report in the radiology reading context. Similarly, for ultrasound image interpretation in the cardiovascular care context (e.g. echocardiogram acquisition), the image interpretation environment 31 is typically operatively connected with the CVIS 25 to retrieve echocardiogram examination images and to enable entry of an image examination findings report, which may be referred to as an echocardiogram report in this context.

To provide finding object-triggered automated access to relevant patient information stored in the electronic patient record 24, 25, 26, as disclosed herein, the non-transitory storage medium also stores finding object detection instructions 32 which are readable and executable by the electronic processor 20, 22 to monitor the image interpretation environment 31 implemented by the image interpretation environment instructions 30 so as to detect generation of a finding object or user selection of a finding object via the at least one user input device 14, 16, 18. The non-transitory storage medium also stores patient record retrieval instructions 34 which are readable and executable by the electronic processor 20, 22 to identify and retrieve patient information relevant to a finding object detected by the finding object detection instructions 32 from at least one electronic patient record 24, 25, 26. Still further, the non-transitory storage medium stores patient record display instructions 36 which are readable and executable by the electronic processor 20, 22 to display patient information retrieved by the patient record retrieval instructions 34 on the at least one display 12 and in the image interpretation environment 31 implemented by the image interpretation environment instructions 30.

In the following, some illustrative embodiments of these components are described in further detail.

The image interpretation environment instructions 30 implement the image interpretation environment 31 (e.g. a radiology reading environment, or an ultrasound image interpretation environment). The image interpretation environment 31 performs operations in accord with user inputs received via the at least one user input device 14, 16, 18 including display of medical images on the at least one display 12, manipulation of displayed medical images (e.g. at least pan and zoom of displayed medical images, and optionally other manipulation such as applying a chosen image filter, adjusting the contrast function, contouring organs, tumors, or other image features, and/or so forth), and construction of an image examination findings report 40. To construct the findings report 40, the image interpretation environment 31 provides for the generation of findings.

More particularly, the image interpretation environment 31 provides for automated or semi-automated generation of standardized and/or structured finding objects. In some radiology workstation environments, finding objects are generated in a standardized Annotation Image Mark-up (AIM) format. In one contemplated user interface, the user selects an image location, such as a pixel of a computed tomography (CT), magnetic resonance (MR), or other radiology image, which is at or near a relevant finding (e.g., a tumor or aneurysm). This brings up a contextual AIM graphical user interface (GUI) dialog 42 (e.g. as a pop-up user dialog box), via which the image interpreter labels the finding with meta-data (i.e. an annotation) characterizing its size, morphology, enhancement, pathology, and/or so forth. These data form the finding object in AIM format. It is to be appreciated that AIM is an illustrative standard for encoding structured finding objects. Alternative standards for encoding structured finding objects are also contemplated. In general, the structured finding object preferably encodes “key-value” pairs specifying values for various data fields (keys), e.g. “anatomy=lung” is an illustrative example in AIM format, where “anatomy” is the key field and “lung” is the value field. In the AIM format, key-value pairs are hierarchically related through a defining XML standard. Other structured finding object formats can be used to similarly provide structure for representing finding objects, e.g. as key-value tuples of a suitably designed relational database table or the like (optionally with further columns representing attributes of the key field, et cetera).

In another illustrative example, suitable for an echocardiogram interpretation environment, the user interface responds to clicking a location on the image by bringing up a point-and-click finding code GUI dialog 44 via which the image interpreter can select the appropriate finding code, e.g. from a contextual drop-down list. Each finding code (FC) is a unique and codified observational or diagnostic statement about the cardiac anatomy, e.g. the finding code may be a word or phrase describing the anatomy feature.

The generation or user selection of the finding object is leveraged in embodiments disclosed herein to trigger a patient record retrieval operation, and the standardized and/or structured finding object provides the informational basis for this retrieval operation. The generation of a finding object (or, alternatively, the user selection of a previously created finding object) is detected by the FO detection instructions 32, so as to generate a selected finding object (FO) 46. The detection can be triggered, for example, by detecting the user operating a user input device 14, 16, 18 to close the FO generation (or editing) GUI dialog 42, 44. Depending upon the particular format of the finding object, some conversion may optionally be performed to generate the FO 46 as a suitable informational element for searching the electronic patient record 24, 25, 26. For example, in the case of a finding object in AIM or another structured format, a medical ontology 48 may be referenced to convert the finding object to a natural language word or phrase. For example, an ontology such as SNOMED or RadLex may be used for this purpose.

The patient record retrieval instructions 34 execute on the server computer 22 to receive the FO 46 and to use the informational content of the FO 46 to identify and retrieve patient information relevant to a FO 46 from at least one electronic patient record 24, 25, 26. In one approach, a non-transitory storage medium stores a relevant patient information look-up table 50 that maps finding objects to patient information items. The look-up table 50 may be stored on the same non-transitory storage medium that stores some or all of the instructions 30, 32, 34, 36, or may be stored on a different non-transitory storage medium. The term “information item” as used in this context refers to an identification of a database field, search term, or other locational information sufficient to enable the executing patient record retrieval instructions 34 to locate and retrieve certain relevant patient information. For example, if the FO indicates a lung tumor, relevant patient information may be whether the patient is a smoker or a non-smoker—accordingly, the look-up table for this FO may include the location of a database field in the EMR or EHR 24 containing that information. Similarly, the look-up table 50 may include an entry locating information on whether a histopathology examination has been performed to assess lung cancer, and/or so forth. As another example, in the case of the finding “Septum is thickened” in the context of an echocardiogram interpretation, the look-up table 50 may include the keywords “hypertrophic cardiomyopathy” and “diabetes” as these conditions are commonly associated with a thickened septum, and the electronic patient record 24, 25, 26 is searched for occurrences of these terms. If the content of the electronic patient record is codified using an ontology such as the International Classification of Diseases version 10 (ICD10), Current Procedural Terminology (CPT) or Systematized Nomenclature of Medicine (SNOMED), then these terms are suitably employed in the look-up table 50. In such a case, the look-up table 50 may further include an additional column providing a natural language word or phrase description of the ICD-10 code or the like. A mapping is maintained between AIM-compliant objects and the history items, e.g., ICD10. The mapping provided by the look-up table 50 may for some entries involve partial objects, meaning that they need not be fully specified. A sample look-up table entry for radiology reading might be:
Finding object: Anatomy=lung and morphology=nodule
Relevant history: “F17—Nicotine dependence”
   while a sample look-up table entry for an echocardiogram interpretation mapping between FCs and the patient record items might be:
Finding object: “Septum is thickened”
Relevant history: “142.2—Hypertrophic cardiomyopathy”
   The electronic patient record retrieval instructions 34 are executable by the electronic processor 22 to identify and retrieve patient information relevant to a finding object 46 by referencing the relevant patient information look-up table 50 for the locational information and then searching the electronic patient record 24, 25, 26 for relevant patient information at that location (e.g. specified as a specific database field, or as a search term to employ in a SQL query or the like, or so forth).

In a variant embodiment, a background mapping is deployed from FCs onto ontology concepts, as the FC are contained in an unstructured “flat” lexicon. Such a secondary mapping can be constructed manually or generated automatically using a concept extraction engine, e.g. MetaMap.

In the following, an illustrative implementation of the electronic patient record retrieval instructions 34 is described. The executing instructions 34 have access to one or more repositories of potentially heterogeneous medical documents and data. The Electronic Medical (or Health) Record (EMR or EHR) 24 is one instance of such a repository. The data sources can have multiple forms, for example: list of ICD10 codes (e.g., problem list, past medical history, allergies list); list of CPT codes (e.g., past surgery list); list of RxNorm codes (e.g., medication list); discrete numerical data elements (e.g., contained in lab reports and blood pressures); narrative documents (e.g., progress, surgery, radiology, pathology and operative reports); and/or so forth. With each clinical condition, zero or more dedicated search modules are defined, each searching one type of data source. For instance, with the clinical condition “diabetes” search modules can be associated that: match a list of known ICD10 diabetes codes against the patient's problem list; match a list of medications known to be associated with diabetes treatment (e.g., insulin) against the patient's medication list; match a glucose threshold against the patient's most recent lab report; match a list of key words (e.g., “diabetes”, “DM2”, “diabetic”) in narrative progress reports; and/or so forth. If there are matches, the executing electronic patient record retrieval instructions 34 return pointers to the location(s) in the matching source document(s) as well as matching elements of information. In one implementation, the executing electronic patient record retrieval instructions 34 perform a free-text search based on a search query derived from the finding object 46, e.g., “lower lobe lung nodule”. This search can be implemented using various search methods, e.g., elastic search. If only FO-based text-based searches are employed, then the relevant history look-up table 50 is suitably omitted. In other embodiments, free-text searching using the words or phrases of the finding object 46 augments retrieval operations using the relevant history look-up table 50.

The identified and retrieved patient history is displayed on the at least one display 12 by the executing patient record display instructions 36. Preferably, the retrieved patient information is displayed on the at least one display 12 and in the image interpretation environment 31, e.g. in a dedicated patient history window of the image interpretation environment 31, as a pop-up window superimposed on a medical image displayed in the image interpretation environment 31, or so forth. In this way, the user does not need to switch to a different application running on the electronic processor 20 (e.g., a separate electronic patient record interfacing application) in order to access the retrieved patient information, and this information is presented in the image interpretation environment 31 that the image interpreter is employing to view the medical images being interpreted. In some embodiments (described later below), relevance learning instructions 52 are readable and executable by the electronic processor 20, 22 to update the relevant patient information look-up table 50 by applying machine learning to user interactions with the displayed patient information via the at least one user input device 14, 16, 18.

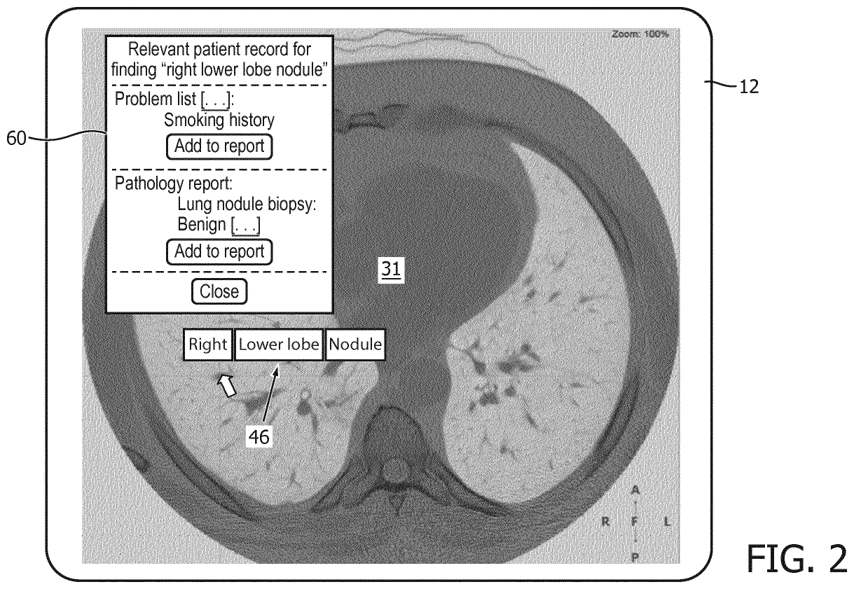

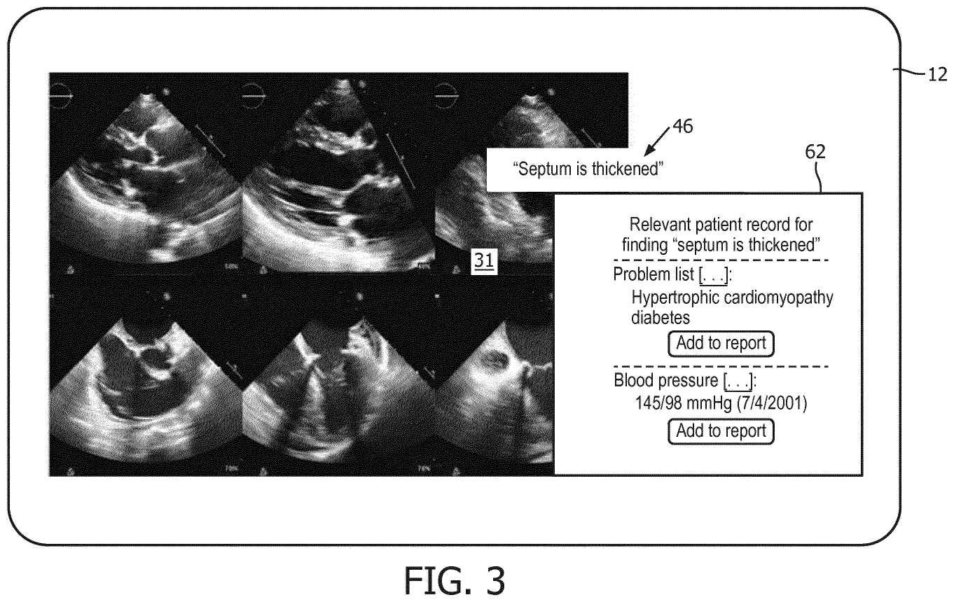

With reference to FIGS. 2 and 3, in some embodiments the executing patient record display instructions 36 are interactive, e.g., by clicking on a particular piece of displayed patient information, a panel appears that displays the source document (e.g., the narrative report) highlighting the matching information and its surrounding content. FIG. 2 illustrates a contemplated display in which the image interpretation environment 31 is a radiology reading environment. The finding object 46 in this example is “right lower lobe nodule”) and is created (e.g. using the AIM GUI dialog 42, or more generally a GUI dialog for entering the finding in another structured format so as to create a structured finding object) and detected by the executing FO detection instructions 32 which monitor the image interpretation environment 31 for generation or user selection of FOs. This detection of the FO 46 triggers execution of the patient record retrieval instructions 34, and the retrieval of relevant patient information then triggers execution of the patient record display instructions 36 to display of relevant clinical history in a pop-up window 60 in the illustrative example of FIG. 2. The underlined elements [ . . . ] shown in the window 60 indicate dynamic hyperlinks that will open up the source document centered at the matching information. Additionally, the window 60 includes “Add to report” buttons which can be clicked to add the corresponding patient information to the image examination finding report 40 (see FIG. 1). A “Close” button in the window 60 can be clicked to close the patient information window 60.

FIG. 3 illustrates a contemplated display in which the image interpretation environment 31 is an echocardiogram image interpretation environment. The finding object 46 in this example is “Septum is thickened” and is created (e.g. using the FC GUI dialog 44) and detected by the executing FO detection instructions 32 which monitor the image interpretation environment 31 for generation or user selection of FOs. This detection of the FO 46 triggers execution of the patient record retrieval instructions 34, and the retrieval of relevant patient information then triggers execution of the patient record display instructions 36 to display of relevant clinical history in a separate patient information window 62 of the image interpretation environment 31. The underlined elements [ . . . ] shown in the window 62 indicate dynamic hyperlinks that will open up the source document centered at the matching information. Additionally, the window 62 includes “Add to report” buttons which can be clicked to add the corresponding patient information to the image examination finding report 40 (see FIG. 1). A “Close” button in the window 62 can be clicked to close the patient information window 62.

With returning reference to FIG. 1, the relevance learning instructions 52 are readable and executable by the electronic processor 20, 22 to update the relevant patient information look-up table 50 by applying machine learning to user interactions with the displayed patient information via the at least one user input device 14, 16, 18. For example, if the user clicks on one of the “Add to report” buttons in the window 60 of FIG. 2 (or on one of the “Add to report” buttons in the window 62 of FIG. 3), this may be taken as an indication that the image interpreter has concluded the corresponding patient information that is added to the image examination findings report 40 was indeed relevant in the view of the image interpreter. By contrast, any piece of patient information which is not added to the report 40 by selection of its corresponding “Add to report” button was presumably not deemed to be relevant by the image interpreter. These user interactions therefore enable the pieces of patient information to be labeled as “relevant” (if the corresponding “Add to report” button is clicked) or “not relevant” (if the corresponding “Add to report” button is not clicked), and these labels can then be treated as human annotations, e.g. as ground-truth values. The executing relevance learning instructions 52 then update the relevant patient information look-up table 50 by applying machine learning to these user interactions, e.g. by removing any entry of the look-up table 50 that produces pieces of patient information whose “relevant”-to-“not relevant” ratio is below some threshold based on the interactions. To enable addition of new information, it is contemplated to statistically add additional information in the patient information retrieval that is not part of the look-up table 50—if these additions are selected by the user as “relevant” with statistics above a certain threshold then they may be added to the look-up table 50. These are merely illustrative examples, and other machine learning approaches may be used.

In the illustrative embodiment, execution of the various executable instructions 30, 32, 34, 36 is distributed between the local workstation computer 20 and the remote server computer 22. Specifically, in the illustrative embodiment the image interpretation environment instructions 30, the finding object detection instructions 32, and the patient record display instructions 36 are executed locally by the local workstation computer 20; whereas, the patient record retrieval instructions 34 are executed remotely by the remote server computer 22. This is merely an illustrative example, and the instructions execution may be variously distributed amongst two or more provided electronic processors 20, 22, or there may be a single electronic processor that performs all instructions.

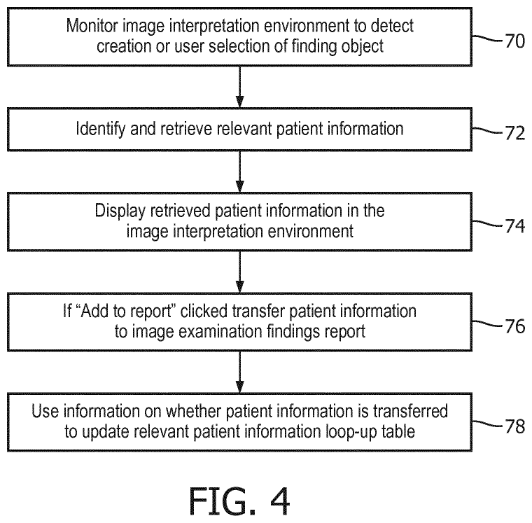

With reference to FIG. 4 and continuing reference to FIG. 1, an illustrative image interpretation method suitably performed by the image interpretation workstation of FIG. 1 is described. In an operation 70, the image interpretation environment 31 is monitored to detect creation or user selection of a finding object 46. In an operation 72, the relevant patient information is identified and retrieved from the electronic patient record 24, 25, 26, e.g. using the relevant patient information look-up table 50. In an operation 74, the retrieved relevant patient information is displayed in the image interpretation environment 31. In an optional operation 76, if the patient clicks on the “Add to report” button in the window 60, 62 of respective FIGS. 2 and 3, or otherwise selects to add a piece of retrieved patient information to the image examination findings report 40, then this information is added to the report 40. In an optional operation 78, the user interaction data on which pieces of retrieved patient information are actually added to the image examination findings report 40 is processed by machine learning to update the relevant patient information look-up table 50.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An image interpretation workstation comprising:
at least one display;
at least one user input device;
an electronic processor operatively connected with the at least one display and the at least one user input device; and
a non-transitory storage medium storing:
image interpretation environment instructions readable and executable by the electronic processor to perform operations in accord with user inputs received via the at least one user input device including display of medical images on the at least one display, manipulation of displayed medical images, generation of finding objects, and construction of an image examination findings report;
finding object detection instructions readable and executable by the electronic processor to detect generation of a finding object or user selection of a finding object via the at least one user input device;
patient record retrieval instructions readable and executable by the electronic processor to identify and retrieve patient information relevant to a finding object detected by the finding object detection instructions from at least one electronic patient record; and
patient record display instructions readable and executable by the electronic processor to display patient information retrieved by the patient record retrieval instructions on the at least one display;
wherein:
execution of the patient record retrieval instructions is automatically triggered by detection of generation or user selection of a finding object by the executing finding object detection instructions; and
execution of the patient record display instructions is automatically triggered by retrieval of patient information relevant to a finding object by the executing patient record retrieval instructions.

2. The image interpretation workstation of claim 1 wherein:
the non-transitory storage medium further stores a relevant patient information look-up table that maps finding objects to patient information items; and
the patient record retrieval instructions are executable by the electronic processor to identify and retrieve patient information relevant to a finding object by referencing the relevant patient information look-up table.

3. The image interpretation workstation of claim 2 wherein the non-transitory storage medium further stores:
relevance learning instructions readable and executable by the electronic processor to update the relevant patient information look-up table by applying machine learning to user interactions with the displayed patient information via the at least one user input device.

4. (canceled)

5. The image interpretation workstation of claim 1 wherein the patient record display instructions are further readable and executable by the electronic processor to detect selection via the at least one user input device of displayed patient information retrieved by the patient record retrieval instructions and to display a corresponding source document retrieved from at least one electronic patient record.

6. The image interpretation workstation of claim 1 wherein:
the image interpretation environment instructions are executable to generate finding objects in a structured format by operations including user selection of an image feature of a displayed medical image via the at least one user input device and input of at least one finding annotation via interaction of the at least one user input device with a graphical user interface dialog; and
the finding object detection instructions are executable by the electronic processor to detect a finding object in the structured format and to convert the finding object in the structured format to a natural language word or phrase using a medical ontology.

7. The image interpretation workstation of claim 1 wherein:
the image interpretation environment instructions are executable to generate a finding object by user selection of a finding code via interaction of the at least one user input device with a graphical user interface dialog; and
the finding object detection instructions are executable by the electronic processor to detect generation or user selection of a finding code.

8. The image interpretation workstation of claim 1 wherein:
the patient record display instructions are further readable and executable by the electronic processor to copy patient information retrieved by the patient record retrieval instructions to the image examination findings report in accord with user inputs received via the at least one user input device.

9. The image interpretation workstation of claim 1 wherein the image interpretation environment instructions are executable to perform manipulation including at least pan and zoom of displayed medical images in accord with user inputs received via the at least one user input device.

10. A non-transitory storage medium storing instructions readable and executable by an electronic processor operatively connected with at least one display and at least one user input device to perform an image interpretation method comprising:
providing an image interpretation environment to perform operations in accord user inputs received via the at least one user input device including display of medical images on the at least one display, manipulation of displayed medical images, generation of finding objects, and construction of an image examination findings report;
monitoring the image interpretation environment to detect generation or user selection of a finding object;
identifying and retrieving patient information relevant to the generated or user-selected finding object from at least one electronic patient record; and
displaying the retrieved patient information on the at least one display and in the image interpretation environment;
wherein:
the identifying and retrieving of patient information is automatically triggered by detection of the generated or user selected finding object; and
the displaying of the retrieved patient information is automatically triggered by the retrieval of the patient information.

11. The non-transitory storage medium of claim 10 further storing a relevant patient information look-up table mapping finding objects to patient information items, wherein the identifying and retrieving of patient information operates by referencing the relevant patient information look-up table.

12. The non-transitory storage medium of claim 10, wherein the image interpretation method further comprises:
updating the relevant patient information look-up table by applying machine learning to user interactions with the displayed patient information via the at least one user input device.

13. (canceled)

14. The non-transitory storage medium of claim 10 wherein the displaying of the retrieved patient information further comprises:
detecting selection via the at least one user input device of selected displayed patient information; and
displaying a source document retrieved from at least one electronic patient record and containing the selected displayed patient information.

15. The non-transitory storage medium of claim 10 wherein:
the image interpretation environment generates finding objects in an Annotation Image Mark-up format by operations including user selection of an image feature of a displayed medical image via the at least one user input device and input of at least one finding annotation via the at least one user input device interacting with a graphical user interface dialog; and
the monitoring of the image interpretation environment to detect generation or user selection of a finding object includes detecting a finding object in the AIM format and converting the finding object in the AIM format to a natural language word or phrase using a medical ontology.

16. The non-transitory storage medium of claim 10 wherein:
the image interpretation environment generates finding objects by user selection of a finding code via interaction of the at least one user input device with a graphical user interface dialog; and
the monitoring of the image interpretation environment to detect generation or user selection of a finding object includes detecting generation or user selection of a finding code.

17. The non-transitory storage medium of claim 10 wherein the manipulation of displayed medical images provided by the image interpretation environment includes at least pan and zoom of displayed medical images in accord with user inputs received via the at least one user input device.

18. An image interpretation method performed by an electronic processor operatively connected with at least one display and at least one user input device, the image interpretation method comprising:
providing an image interpretation environment to perform operations in accord user inputs received via the at least one user input device including display of medical images on the at least one display, manipulation of displayed medical images, generation of finding objects, and construction of an image examination findings report;
monitoring the image interpretation environment to detect generation or user selection of a finding object;
identifying and retrieving patient information relevant to the generated or user-selected finding object from at least one electronic patient record; and
displaying the retrieved patient information on the at least one display and in the image interpretation environment.

19. The image interpretation method of claim 18 wherein the identifying and retrieving of patient information operates by referencing a relevant patient information look-up table that maps finding objects to patient information items.

20. The image interpretation method of claim 18 wherein:
the image interpretation environment generates finding objects in an Annotation Image Mark-up format by operations including user selection of an image feature of a displayed medical image via the at least one user input device and input of at least one finding annotation via interaction of the at least one user input device with a graphical user interface dialog; and
the monitoring of the image interpretation environment to detect generation or user selection of a finding object includes detecting a finding object in the AIM format and converting the finding object in the AIM format to a natural language word or phrase using a medical ontology.

21. The image interpretation method of claim 18 wherein:
the image interpretation environment generates finding objects by user selection of a finding code via interaction of the at least one user input device with a graphical user interface dialog; and
the monitoring of the image interpretation environment to detect generation or user selection of a finding object includes detecting generation or user selection of a finding code.
